(19) United States
(12) Patent Application Publication
(10) Pub. No.: US 2003/0124640 A1
(43) Pub. Date: Jul. 3, 2003

(54) Predictive assay for the outcome of IVF

(75) Inventors: Brian Cooke, London (GB); Anthony Michael, London (GB)

Correspondence Address: NIXON & VANDERHYE, PC, 1100 N GLEBE ROAD, 8TH FLOOR, ARLINGTON, VA, 22201-4714, US

(73) Assignee: Royal Free Hospital School of Medicine

(21) Appl. No.: 10/322,474

(22) Filed: Dec. 19, 2002

Related U.S. Application Data
(63) Continuation of application No. 09/125,254, filed on Feb. 9, 1999, now abandoned.
Application No. 09/125,254 is a 371 of international application No. PCT/GB97/00445, filed on Feb. 17, 1997.

(30) Foreign Application Priority Data
Feb. 16, 1996 (GB) 96 03326.1

(51) Int. Cl. C12Q 1/26; G06F 19/00; G01N 33/48; G01N 33/50

(52) U.S. Cl. 435/25; 702/19

(57) ABSTRACT

The present invention relates to assays (and kits) for predicting the chances of success of pregnancy using in vitro fertilisation (IVF) by determining the level of a modulator of 11β-hydroxysteroid dehydrogenase (11β-HSD) in a biological sample taken from a human female, for example in the environment of an oocyte, e.g. follicular fluid or granulosa cells. The amount of modulator (e.g. inhibitor) can be determined by reference to its effect on 11β-HSD activity and administration of a modulator (e.g. an 11β-HSD inhibitor) may increase the likelihood of pregnancy while 11β-HSD agonists may be potential contraceptive agents.

The present invention relates to an assay which can be used to assess the likelihood of pregnancy (or infertility) in a female, and especially is predictive of the outcome of in vitro fertilisation (IVF) in mammals (including humans). In particular it relates to an assay for modulators (such as inhibitors) of 11β-hydroxysteroid dehydrogenase (11β-HSD) in tissue or fluid taken from the environment of a developing (or developed) oocyte.

The technique of IVF has been used in human patients with infertility problems since 1978. Despite extensive research it is still a difficult procedure, and even in the best IVF clinics a success rate of only 30% is generally achieved.

IVF is an expensive procedure and can be psychologically traumatic for a patient. Surgical procedures are required to collect eggs from a female for IVF and, following fertilization, further surgery is required to implant the fertilised eggs in the womb. The recipient must then wait for a period of time before it can be determined whether or not pregnancy has been established. In some cases, pregnancy may never be achieved despite repeated attempts, and these cases can represent a considerable expense to the patient and society, both in financial and human terms.

Therefore, until success rates of IVF can be improved, it would be desirable to be able to identify recipients for whom IVF is unlikely to be successful prior to treatment, so that such patients may avoid the above mentioned costs and trauma of the IVF procedure.

The adrenal steroid hormone, cortisol, is believed to influence maturation of the female germ cell (the oocyte) and the development of ovarian cells in culture ^{22, 23}. Recently, it has been reported that there is an association between the concentration of cortisol in follicular fluid and oocyte maturity.

The enzyme 11β-hydroxysteroid dehydrogenase (11β-HSD, EC 1.1.1.146) converts cortisol and corticosterone to their inactive forms, cortisone and 11β-dehydrocorticosterone, respectively.^{3,5,16} It is present in rat oocytes³ and appears to modulate ovarian function.¹² Two isoforms of 11β-HSD have been characterised; a hepatic form and a renal form. Both isoforms are expressed in ovarian tissue. In this specification, reference to 11β-HSD includes both (hepatic and renal) isoforms (unless the context requires otherwise).

Previous studies have tended to focus upon the relationship between oocyte maturity or hormone levels and the success of fertilization in vitro. However, it was surprisingly found that once fertilisation has been achieved and the second part of the IVF procedure is performed, namely implantation, there was a strong inverse correlation between levels of 11β-HSD in the environment of the oocyte at the time of collection and the subsequent establishment of pregnancy. This correlation exists regardless of the maturity of the oocyte or other factors which may affect fertilization.^{13,15,37} This lead to a method of predicting the outcome of IVF which involved firstly determining the level of 11β-HSD in a biological sample taken from a female patient and then predicting, from the level of 11β-HSD determined, the probability of establishing pregnancy in the subject by IVF³⁷. One could then screen female subjects for their suitability to take part in IVF programs.

It has now been discovered that 11β-HSD activity can vary widely between follicles taken from the same individual. The activity measured for a pool of cells from different follicles (from the same individual) was not always a true reflection of activity in individual follicles, suggesting that one or more follicles possess 11β-HSD modulator(s) affecting the results of the entire pool. It thus appears that IVF outcome may be predicted on the basis of these modulators (by their presence and/or activity).

The present invention thus relates to assay methods and assay kits which can be used to assess the likelihood of pregnancy (or, more accurately, successful implantation), and in particular to predict the outcome of IVF in a female individual. The invention also relates to such methods and kits for use in a method of diagnosis in order to determine the outcome of IVF or the suitability of a female individual for IVF treatment. Although the invention has been developed from research on human females, it is applicable to any mammalian female and can be used to increase the success of, for example, captive breeding programmes of endangered species or commercial breeding by IVF of livestock such as cattle or horses.

Thus a first aspect of the invention comprises a method of predicting the outcome of, or assessing the likelihood of success of, IVF, which method comprises:

(a) determining the level of a modulator of the enzyme 11β-hydroxysteroid dehydrogenase (11β-HSD) in one or more biological sample(s) from a female individual; and

(b) predicting, from the level(s) of the modulator determined, the likelihood or probability of establishing pregnancy in that individual by IVF.

The (or each) sample may comprise a body fluid or a tissue, such as from the environment of the oocyte. This can comprise granulosa-lutein cells or follicular cells as well as other ovarian cells, recovered for example from the ovarian follicles of women undergoing oocyte recovery for in vitro fertilization and embryo transfer. Alternatively, the (or each) sample may comprise a follicular aspirate, for example obtained on an out-patient basis prior to admission to an IVF programme. The sample may also comprise stored (usually frozen) cells from the environment of an oocyte. Other samples can comprise urine or plasma. A particularly preferred sample comprises follicular fluid.

The first aspect may thus additionally comprise, prior to (a), removing one or more biological sample(s) from the female.

The sample may be treated (eg. stored, frozen, washed, cultured, added to a medium, etc) before the determination in (a) is conducted.

By “modulator” of 11β-HSD is meant a substance (whether naturally occurring or not) that affects 11β-HSD activity. Preferably the modulator is endogenous. This may mean increasing 11β-HSD activity (an agonist, or cofactor) or decreasing activity (an inhibitor), as well as altering the enzyme's specificity or physiological properties.

Suitable 11β-HSD inhibitors include glycyrrhetinic acid, glycyrrhetinic acid-like factor (GALF), gossypol and bioflavonoids, as well as glucocorticoid hormones or analogues thereof (which can compete with cortisol for 11β-HSD). Other compounds that modulate 11β-HSD activity include bile salts, cholesterol and steroid hormones (pregnenolone and progesterone both inhibit). Preliminary studies suggest that oestradiol may, in some cases, act as an inhibitor.

Inhibitory activity and site of action can vary. For example, studies have shown that pregnenolone and progesterone both inhibit renal and ovarian 11β-HSD activities acutely. Also, it appears that oestradiol inhibits hepatic 11β-HSD activity in vivo, but seems to have no acute effect on ovarian 11β-HSD activity in vitro.

The level of the 11β-HSD modulator may be measured directly (eg. by determining the amount or concentration of the modulator) or indirectly (eg. by the level of 11β-HSD activity, as that will often be affected by the modulator).

Direct methods can thus include chromatography (TLC or HPLC) while indirect methods may include measuring the effect of any modulator in the sample by, for example, adding 11β-HSD, a substrate of the enzyme (eg. ³H-cortisol) and determining the affect, if any, on the enzyme's activity. Alternatively or in addition the sample (eg follicular fluid) may be contacted with 11β-HSD present in another body fluid, or cultured cells (eg human granulosa-lutein cells) or other body-derived substances (eg homogenised animal (eg rat) organs, such as kidneys). A parallel, control, assay may also need to be conducted to allow for any 11β-HSD already (and naturally) present in the sample. The 11β-HSD used in the assay may be from an isolated (or purified) source or can be present in another (human or animal) body or body-derived fluid. The latter includes, for example, (eg human) granulosa-lutein cells and organ (eg kidney) homogenates. The 11β-HSD used in the assay may not necessarily be human, it may, for example be from an animal species, such as from a rodent (eg rat). This can allow assays to be performed using relatively cheap and accessible forms of 11β-HSD (eg rat kidney homogenates).

In another assay the sample (eg follicular fluid) to be assayed can be removed along with an 11β-HSD source (eg granulosa-lutein cells). They are then separated, for example the cells cultured for several days, as a control, in the absence of bodily fluids, and then contacted (i.e. reunited) with the fluid. The effect on 11β-HSD levels can then be assayed. This type of assay uses local fluids/cells and so can be used to test for the presence of a modulator in the sample (follicular fluid).

In determining the level of 11β-HSD modulator the process may comprise repeating the determination in (a) for:

(i) each of a plurality of samples taken from the same female individual; and

(ii) a mixture (or pool) of at least two of those samples.

A comparison can then be made between the levels found in (i) and (ii). A statistical (or significant) difference suggests that one sample in (i) possesses a modulator which affects the result found for the pool of samples (ii). Thus, if the level found for (ii) is less than the mean of the individual samples each determined in (i) this may indicate the presence of an 11β-HSD inhibitor in one of the samples. This difference can thus be used as a measurement of the level of modulator in a sample, and is easily calculated from the formula:

$$Δ ( Difference ) = \frac{{A}_{1} + {A}_{2} + {A}_{3} + … + {A}_{n} - {A}_{m}}{n}$$

where Aₓ is the amount of modulator (determined directly or indirectly by 11β-HSD activity) for sample x, determined in (i), for n samples, and Aₘ is the modulator level in the mixture (or pool) of (from 2 to n) samples determined in (ii). Here, each sample is preferably taken from a different follicle (or from the environment of a oocyte), and can comprise follicular fluid and/or granulosa cells.

It will therefore be apparent that a positive value of A indicates the presence of an inhibitor (of 11β-HSD in one of the samples), while conversely a negative value suggests the presence of an agonist. The magnitude of A will thus give an indication as to the amount of modulator present and indeed is likely to be directly proportional to the concentration of the modulator. Thus a positive value of A would indicate a greater probability of pregnancy. Hence where the invention refers to the level(s) of 11β-HSD modulator determined (for example in predictions of likelihood or probability of establishing pregnancy) that can include calculating A and using that value as a basis for any predictions or clinical evaluations.

In addition the determination in (a) may be made for two or more biological samples (taken at) different times (eg. in the controlled ovarian hyperstimulation cycle), and any difference noted. Furthermore the biological samples may be taken from the individual at the same (or similar) time in different cycles. Note that reference is made here to controlled ovarian hyperstimulation cycles because women undergoing preparation for oocyte collection neither menstruate nor ovulate.

An increase (or decrease) in A can thus point to a progressive change in amount (or effect) of the modulator and may give an indication on whether IVF is to be successful. For example, samples may be taken on successive days, and a progressive decline in 11β-HSD activity (as opposed to no change, or an increase) may indicate a greater chance of pregnancy.

The level of 11β-HSD activity can, in turn, also be determined directly or indirectly. That is to say that the level of 11β-HSD may be measured as an amount (of the protein) or in terms of its activity. Direct methods include enzyme assays to determine the level of 11β-HSD activity which involve contacting the sample with a substrate, for example ³H-cortisol, and measuring the conversion of the substrate (eg. to ³H-cortisone) by the enzyme. ³H-cortisol and ³H-cortisone can be separated by thin layer chromatography and then quantified. This will provide a direct measurement of enzyme activity, and for this reason is preferred. In a typical assay, a concentration of about 100 nM of ³H-cortisol may be used, although a concentration ranging from 10 nM to 1000 nM or more can be used.

Indirect methods of measuring 11β-HSD activity include measuring the levels of cortisol and cortisone in the sample, and determining the ratio of the two as an indirect measure of enzyme (or modulator) activity. In such a case, the higher the level of cortisone in relation to cortisol, the higher the activity of the enzyme (or low level of inhibitor). The levels of cortisol and cortisone can be measured by methods known per se (eg by immunoassay methods having resolved cortisol and cortisone by TLC/HPLC). Kits for the assay of cortisol are commercially available.³⁴

Alternatively, 11β-HSD levels can be measured by immunoassay or similar (eg. competitive) ligand-binding techniques. This will provide an indication of the amount of the enzyme, which may be correlated to enzyme activity (and from there to modulator activity). For example, a ligand (or antibody) capable of binding the enzyme could be used in immunoassay methods such as RIA or ELISA. Methods to determine and obtain ligands which bind with high affinity to a specific analyte are also available in the art.³⁵ In addition, the level of 11β-HSD protein, or even its MRNA, can be used as a measure of modulator activity since some modulators (eg. oestradiol) exert their effect at the level of MRNA transcription or translation.

The expression of the 11β-HSD enzyme can also be measured by immunocytochemistry using a monoclonal antibody. Such techniques will provide a measurement of the amount of 11β-HSD present, which can then be correlated to enzyme activity.

Although reference is made in this specification to determining levels of 11β-HSD (and its modulators) it will be understood from the foregoing that this also includes the indirect measurements mentioned above.

Once the level of modulator (or 11β-HSD activity) has been measured, the result can be used to predict or assess the likelihood of successful establishment of pregnancy in a female subject undergoing IVF treatment. The level of 11β-HSD activity in the sample will be directly affected by the modulator. Therefore, 11β-HSD activity will be proportional (or inversely proportional) to the level of agonist (or antagonist/inhibitor) and a measurement of the level of the modulator can thus be correlated back to (or provide an indication of) the level of 11β-HSD activity. In previous studies 11β-HSD activity levels have been measured by the amount of cortisol converted to cortisone per mg protein per 4 hours to obtain a direct measurement of enzyme activity. Those subjects with enzyme activities less than about 10 pmol/mg/4 hr had a pregnancy rate of over 65% following embryo transfer. In contrast, subjects. with enzyme activities that ranged from 15 to 111 pmol/mg/4 hr did not become pregnant even though fertilization of their oocytes was apparently successful.³⁷ Thus levels of (11β-HSD) modulator can be used to predict the chances of pregnancy (in particular, the probability of pregnancy during IVF).

Those of skill in the art will appreciate that although recent research has determined a “cut-off” level of 11β-HSD activity above which patients have not become pregnant (and below which patients have significantly improved probability of successful pregnancy), the value is a statistical measure and other measurements and thresholds can be used. A corresponding threshold level of 11β-HSD modulators can thus also be arrived at and used for the same purpose. In practising the invention, it is most important to achieve consistency of assay, and so each individual practitioner (or IVF team) will be capable of establishing their own particular assay method and determining their own “cut-off” level. This could be established by first conducting a historical study on samples from previous patients.

Thus, a level of 11β-HSD activity of 10 pmol/mg/4 hr (as mentioned above) represents the measure that has been used in the past as a suitable limit, and may still be used as a threshold in the practise of the present invention, if a practitioner sees fit. However, if levels of 11β-HSD activity (or modulator) were to be measured in any of the other ways mentioned above, it would be desirable to conduct, using routine procedures, a control using our method of assay in order to determine the relationship between the results and the results of other methods, in order to make direct comparisons.

Once the level of modulator (or 11β-HSD activity) has been determined, it can be used to assess the likelihood of establishing pregnancy by IVF in a patient. For example, the invention can be used in relation to samples from patients who have already had oocytes collected, fertilised in vitro, and implanted. Generally, a number of eggs are collected and fertilised so that in the event of failure to establish pregnancy, more fertilised eggs can be implanted. By conducting the method of the present invention, it is possible to predict, where pregnancy is not established, whether implantation of further stored (fertilised) oocytes is likely to be successful. If levels of modulator (or 11β-HSD activity) in such patients is significantly above the level associated with successful pregnancy, then it would be a saving in time, money and stress to the individual not to undertake further attempts at implantation with stored oocytes collected and fertilised at the same time as those previously implanted and for which modulator (or 11β-HSD activity) data are available.

The methods of the present invention may be performed prior to implantation, prior to fertilization of collected oocytes or even prior to collection of such oocytes. In such cases, the results of such methods may allow the practitioner (or WF clinic) to decide whether or not to even attempt a first implantation.

Thus, a second aspect of the present invention also provides a method for predicting the outcome of IVF in a female individual, the method comprising:

(a) removing or obtaining one or more biological sample(s) from the individual;

(b) determining the level(s) of a modulator of 11β-hydroxysteroid dehydrogenase (11β-HSD) in the sample(s); and

(c) predicting, from the level(s) of 11β-HSD modulator determined, the likelihood or probability of establishing pregnancy in that individual by IVF.

Suitable biological samples include those mentioned for the first aspect. As previously discussed, more than one sample (not necessarily of the same type, but usually so) may be removed. The determination in (b) may then be performed on each sample, and on a mixture (or pool) of samples. Each sample is preferably taken from a different follicle present in the same female.

This embodiment of the invention can be used to select individuals likely to benefit from an IVF programme. Once an individual has been selected, it will be desirable to confirm their suitability during the IVF procedure by repeating assays for 11β-HSD modulators during the initial part of the IVF procedure.

Thus, in a third aspect the invention comprises a method for establishing the likelihood of successful IVF treatment in an individual, the method comprising:

(a1) removing one or more oocyte(s) from a female individual together with a biological sample;

(a2) fertilising an oocyte in vitro;

(b) determining the level of a modulator of 11β-hydroxysteroid dehydrogenase (11β-HSD) in the sample; and

(c) predicting, from the level of 11β-HSD modulator determined, the likelihood or probability of establishing pregnancy in that individual by WF.

In a fourth aspect the invention relates to a method which comprises:

(a) removing one or more oocyte(s) from a female individual together with a biological sample;

(b) determining the level of a modulator of 11β-hydroxysteroid dehydrogenase (11β-HSD) in the sample;

(c1) predicting, from the level of 11β-HSD modulator determined, the likelihood or probability of establishing pregnancy in that individual by IVF; and

(c2) fertilising an oocyte from those individuals whose 11β-HSD modulator level is above or below a predetermined threshold.

Optionally, these embodiments of the invention further comprise:

(d) implanting into the female individual the fertilized oocyte.

Preferred biological samples comprise granulosa-lutein cells and/or follicular fluid.

Although both these embodiments are desirably practised on individuals who have already been assayed prior to oocyte collection for suitable levels of 11β-HSD, they may also be practised on patients who have not undergone such an initial screen.

The invention finds application in large scale screening programs of potential IVF recipients who have been referred to, or present themselves at, IVF clinics. In this (fifth) aspect, the invention comprises:

(a) screening a population of female individuals seeking treatment for infertility by IVF for levels of modulator(s) of 11β-HSD; and

(b) selecting from that population those individuals who have an 11β-HSD modulator level(s) above or below a predetermined threshold for IVF treatment.

In particular, those patients who have modulator levels in the environment of the ovary, especially in granulosa lutein cells, above (if the modulator is an inhibitor), or below (if the modulator is an agonist) a predetermined threshold are preferred as suitable recipients for IVF treatment. This can be seen from the following Table 1:

| | | | | Likelihood of |
|---|---|---|---|---|
| 11β-HSD Activity | Δ | Modulator Level | | Pregnancy |
| Low | +ve | High | (inhibitor) | High |
| | | Low | (agonist) | |
| High | −ve | Low | (inhibitor) | Low |
| | | High | (agonist) | |

Thus the type of modulator, and its amount and/or effect can be used as a predictor for the likelihood of pregnancy, for example the presence of an inhibitor in a female with low 11β-HSD activity may suggest a greater chance of successful IVF.

If the level of modulator(s) is only determined in one cycle then selection (or predictive outcome) may only be possible based on the results for that cycle, although determinations over several cycles may give a more general indication of the outcome of IVF.

By use of the present invention, it will be possible for IVF clinics to allocate resources more efficiently, so that patients with high levels of an 11β-HSD inhibitor (or low levels of an agonist) in the environment of a recovered oocyte, who may thus be unlikely to become pregnant by IVF treatment, are not treated.

The levels of 11β-HSD modulator(s) in a female individual may be monitored over a period of time in order to establish whether or not changes favourable to successful IVF occur. Levels of ovarian 11β-HSD in individual patients can vary between consecutive controlled ovarian hyperstimulation cycles (as well as between follicles in the same cycle). Females may thus be monitored in accordance with the invention to obtain an oocyte which is from an environment with favourable (i.e. low) levels of 11β-HSD (e.g. high levels of 11β-HSD inhibitor or low levels of an agonist).

Since it appears that high levels of 11β-HSD inhibitor (or low levels of an agonist) may increase the chances of pregnancy, one may seek to influence the levels of 11β-HSD to improve the likelihood of successful implantation.

In order to achieve this the invention provides, in a sixth aspect, an identification method, comprising:

(a) obtaining or removing, from a female individual, one or more biological sample(s); and

(b) determining the identity of a modulator (if present) of 11β-HSD in the sample.

Identification may make use of one or more techniques well known in the art, or a combination thereof, such as TLC, HPLC, NMR, IR, mass spectroscopy, etc.

In addition, in a seventh aspect, the present invention relates to a method of increasing the likelihood of pregnancy, the method comprising:

(a) determining the identity of an inhibitor or agonist of 11β-HSD in one or more biological sample(s) from a female individual; and

(b) administering, to that individual, an amount of the inhibitor or an amount of a compound that interferes with, inhibits or prevents the activity of the agonist (a pregnancy enhancing compound). The amount may thereby reduce the level of activity of 11β-HSD.

Conversely, in an eighth aspect of the present invention, the present invention relates to a method of contraception (or decreasing the likelihood of pregnancy), the method comprising:

(a) determining the identity of an inhibitor or agonist of 11β-HSD in one or more biological sample(s) from a female individual; and

(b) administering, to that individual, either an amount of the agonist or an amount of a compound that interferes with, inhibits or prevents the activity of the inhibitor (a contraceptive compound). The amount is such that generally the level of 11β-HSD activity is increased.

The inhibitor or agonist administered in (b) can either be the inhibitor or agonist (after isolation and/or purification) identified in (a) or the same substance, except from a different source. The latter is preferable since the inhibitor or agonist may be administered sterile and/or with other substances such as excipients. Preferably the inhibitor administered is taken from a commercially available source (e.g. Sigma-Aldrich, Poole, Dorset, UK).

A ninth aspect of the present invention thus relates to the use of an 11β-HSD inhibitor or a pregnancy enhancing compound for the manufacture of a medicament for increasing the likelihood of pregnancy in IVF If the inhibitor is progesterone then (since it is administered orally as a contraceptive) it should be given at a dose that will be the same as, or below, physiological level(s) of progesterone.

A tenth aspect of the present invention relates to the use of an 11β-HSD agonist or contraceptive compound (as defined in the eighth aspect) for the manufacture of a contraceptive medicament.

In an eleventh aspect the invention relates to a method of screening potential candidate therapeutic substances. The method may comprise identifying a pregnancy enhancing compound or a contraceptive compound, the method comprising:

(a) bringing into contact an amount of 11β-HSD and a candidate substance; and

(b) measuring the effect, if any, the substance has on modulating 11β-HSD activity;

an inhibition (or decrease) of activity indicating potential pregnancy enhancing properties while an increase in activity indicates potential contraceptive activity.

As will be discussed below, potential 11β-HSD inhibitors include antibodies (or fragments thereof) specific for 11β-HSD (and may thereby reduce or prevent activity by immunoneutralisation).

Females may thus be treated to modulate or block 11β-HSD activity in vivo prior to oocyte recovery. For example, antibodies against, or inhibitors of, 11β-HSD could be administered to, or introduced into, the individual in order to inhibit enzyme activity. This is analogous to methods of treating tumours or other conditions using antibody therapy. For the purposes of this invention, the term “antibody”, unless specified to the contrary, includes fragments of whole antibodies which retain their binding activity for a tumour target antigen. Such fragments include Fv, F(ab′) and F(ab′)₂ fragments, as well as single chain antibodies. Furthermore, the antibodies and fragments thereof may be humanised antibodies, eg. as known in the art.³⁶

Antibodies against 11β-HSD for use in the present invention may be monoclonal or polyclonal antibodies. Monoclonal antibodies may be prepared by conventional hybridoma technology using the proteins or peptide fragments thereof, as an immunogen. Polyclonal antibodies may also be prepared by conventional means which comprise inoculating a host animal, for example a rat or a rabbit, with a peptide of the invention and recovering immune serum.

It may also be possible to inhibit the activity of 11β-HSD using other glucocorticoid hormones or analogues thereof which compete with cortisol for 11β-HSD. Suitable inhibitors of 11β-HSD have already been mentioned (see page 4). Such hormones or analogues thereof can be identified by a screening process where the candidate hormones or analogues thereof are assayed to determine whether they can compete with ³H-cortisol for 11β-HSD, and thus inhibit the activity of the enzyme. Candidate hormones or analogues thereof may then be screened (for in vivo efficacy) by administering an effective amount of a hormone or analogue thereof to female subjects

undergoing IVF treatment who are found to have levels of 11β-HSD which are which are unfavourable to establishing pregnancy following IVF-ET. The amount of inhibitor (eg. hormone or analogue thereof) to be administered will need to be determined by the physician, taking into account its activity and the condition of the patient. This can be achieved without difficulty for hormones since they are often used in clinical practice in fertility clinics. Indeed, it is likely that the inhibitor administered will be naturally occurring, and may already be present in the recipient female. The inhibitor(s) thereof may be administered by any suitable route, e.g. orally or by injection. For administration, the inhibitor may be formulated with a pharmaceutically acceptable carrier or diluent.

In such ways, it may be possible for the practitioner (or IVF team) themselves to modulate the levels of 11β-HSD activity in an individual desiring to undergo IVF, so as to increase the chances of IVF being successful.

The use of 11β-HSD inhibitors may also permit in vitro treatment of collected oocytes to reduce enzyme activity prior to fertilization of oocytes. This may be achieved by bringing into contact an effective amount of such an inhibitor, for example a hormone or analogue thereof as previously mentioned, with a sample comprising an oocyte and surrounding tissue such as the granulosa lutein cells, in order to inhibit the activity of 11β-HSD in the sample. The sample and inhibitor may be brought into contact under sterile conditions such as those typically used for IVF. The use of 11β-HSD inhibitors may also permit in vitro treatment of collected oocytes to reduce enzyme activity prior to fertilization of oocytes. This may be achieved by bringing into contact an effective amount of such an inhibitor, for example a hormone or analogue thereof as previously mentioned, with a sample comprising an oocyte and surrounding tissue such as the granulosa lutein cells, in order to inhibit the activity of 11β-HSD in the sample. The sample and inhibitor may be brought into contact under sterile conditions such as those typically used for IVF.

In a twelfth aspect of the present invention there is provided a method of increasing the likelihood or probability of pregnancy, the method comprising:

(a) optionally removing an oocyte from a female individual; and

(b) treating a removed oocyte with an inhibitor of 11β-HSD or a pregnancy enhancing compound.

Preferably the method additionally comprises:

(c) fertilising the oocyte before, or after, the treatment in (b); and

(d) implanting the fertilised oocyte into a female individual.

The present invention also provides kits for use in performing the assay of the invention. Such kits include at least one reagent useful for the detection of a modulator of 11β-HSD activity. Suitable reagents (for direct detection or determination of modulator concentrations) include antibodies, or other suitable ligand-binding reagents, against the 11β-HSD modulator optionally linked to a label. Typical labels are those commonly used in immunoassay procedures, for example horse radish peroxidase. Alternatively, the kit may contain antibodies, or other suitable ligand-binding reagents, against cortisol and/or cortisone. This may be suitable for indirect assays (for measuring the level of 11β-HSD activity) such as the determination of cortisol: cortisone ratio or radiometric conversion of [³H]-cortisol to [³H]-cortisone. The kit may also contain standards, for examples predetermined amounts of cortisone, cortisol and/or 11β-HSD, any or all of which may be labelled with a detectable label. The kit may also contain enzyme cofactors, for example, NAD or NADP which are converted to NADH or NADPH respectively.

The kit may comprise agents such as oxidised tetrazolium salts to serve as a calorimetric substrate for the re-oxidation of the reduced NAD(P)H. The change in optical density of the indicator salt at the appropriate wavelength (for its reduced form) may be directly proportional to the rate of reduction of the NAD(P)+cofactor, which may in turn be directly proportional to 11β-HSD activity and hence inversely proportional to the concentration of, for example, 11β-HSD inhibitor in the sample.

Alternatively or in addition the kit may contain an 11β-HSD modulator (such as an inhibitor, eg. glycyrrhetinic acid) as a standard for comparison. This may be present in a known concentration (or amount) or at several concentrations (or amounts) so that a calibration curve may be derived for comparison.

The invention also provides a kit for the identification of, or measurement of a level of, a modulator of 11β-HSD (eg. in a sample) for use in a method of diagnosis, prognosis, and/or IVF treatment of a female individual.

The invention also comprises the use of the above mentioned antibodies, fragments and variants thereof, and other suitable ligand-binding reagents, which may optionally be labelled with a detectable label for the manufacture of a diagnostic kit for use in the treatment or diagnosis of suitability for IVF.

Levels of 10-HSD activity may also be assayed via analysis of the levels of 11β-HSD mRNA present in samples obtained. In order to achieve this, 11β-HSD cDNA³⁰ or fragments thereof may be used as a probe to determine levels of 11β-HSD in the environment of the oocyte. Such probes may also be formulated into kits in a manner analogous to those described for antibodies, and may contain control nucleic acids. Probes for the 11β-HSD gene may be designed for use as probes, for example for use in a nucleic acid amplification assay.

Preferred features of one aspect of the invention are applicable to other aspects mutatis murtandis.

The following (non-limiting) examples are provided in order to illustrate the present invention and refer to the accompanying drawings, in which:

FIG. 1 is a graph of 11β-HSD activity against patient number showing the variation of 11β-HSD activities in individual follicles from 12 different patients. The asterisk (*) indicates significant variation between follicles (P<0.05 by ANOVA) for a given patient. (The dotted line at 10 pmol cortisone formed/mg protein.4h indicates the non-specific assay detection limit);

FIG. 2 is a graph of 11β-HSD activity against oocyte maturity showing the relationship between ovarian 11β-HSD activities and oocyte maturity on an individual follicle basis. (The data relate to 34 follicles from 9 different patients; the dotted line at 10 pmol cortisone formed/mg protein.4h indicates the non-specific assay detection limit); and

FIG. 3 is a bar graph of 11β-HSD activity showing the relationship of the 11βHSD activity of a multi-follicular pool of cells (shaded vertical bar) to those activities of the constituent individual follicles (open circles) and the arithmetic mean of the latter individual values (horizontal line) for three different patients. (*P<0.05 for the multi-follicular pool value versus the corresponding arithmetic mean for the individual follicles; unpaired t-tests. The dotted line at 10 pmol cortisone formed/mg protein.4h indicates the non-specific assay detection limit).

### EXAMPLE 1

It has been previously shown that detectable metabolism of cortisol to cortisone by 11β-HSD in human granulosa-lutein cells, pooled for each patient from all aspirated ovarian follicles, is associated with failure to conceive by in vitro fertilization and embryo transfer.¹³ The aims of the experiments detailed here were to assess: (1) the variation in the 11β-HSD activities of granulosa-lutein cells obtained from individual follicles; (2) whether the 11β-HSD activity of pooled granulosa-lutein cells reflects the 11β-HSD activities of the individual follicles for a given patient.

In more detail, in view of the known inverse relationship between ovarian 11 β-HSD activity and the probability of conception by IVF-ET,¹³ one objective was to evaluate the variation in 11β-HSD activities between granulosa-lutein cells from different individual follicles in a given patient, and to appraise the relationship between follicular 11β-HSD activities and oocyte maturity scores. In addition, it was assessed whether the 11β-HSD activity of the multi-follicular pool of cells was equal to the arithmetic mean of those activities in the individual follicles from which that pool was derived. Also determined was whether the 11β-HSD activity of a pool of granulosa-lutein cells combined from several patients differed significantly from the mean of the activities in the multi-follicular pools of cells from each individual patient.

Preparation and Culture of Human Granulosa-Lutein Cells.

Granulosa cells were obtained from patients undergoing assisted conception by IVF-ET following controlled ovarian hyperstimulation as described previously⁸. Follicular aspirates were stored (for up to 3 days) and transported at 4° C. before the preparation of cells. Granulosa cells were isolated from follicular aspirates on 60% (v/v) Percoll (Sigma Chemical Co., Poole, Dorset, UK) and washed repeatedly in Dulbecco's modified phosphate-buffered saline³³ (Life Technologies Ltd., Paisley, Scotland, UK).

Cells were counted by haemocytometer and viability was assessed by the exclusion of 0.4% (v/v) trypan blue dye.

In the first series of experiments (n=12 patients), cells from different individual follicles were isolated on separate Percoll preparations and were diluted to a density of 50,000 viable cells/ml in mixed medium (1:1 Dulbecco's Modified Eagle's Medium:Ham's F-12; Life Technologies Ltd., UK) supplemented with 10% (v/v) foetal calf serum, 2 mmol/1 L-glutamine, 870001U/1 penicillin, and 87 mg/l streptomycin (Life Technologies Ltd., UK). Three 1 ml volumes were then inoculated into a 24-well culture plate to allow for the triplicate assay of 11β-HSD activity in each individual follicle. For 3 of these 12 patients, excess granulosa cells remained after the allocation of ≧150,000 viable cells for the triplicate assay of each follicular 11β-HSD activity. In these latter three cases, equal numbers of cells from each individual follicle were combined to produce a multi-follicular pool of cells where that pool contained ≧150,000 viable cells at a density of 50,000 viable cells/ml. These pooled cells were then plated in three 1 ml volumes in a 24-well plate for the triplicate assay of 11βHSD activity in that multi-follicular pool of cells.

### EXAMPLE 2

In the second series of experiments, the procedure of Example 1 was followed except that all follicular cells aspirated from a given patient (i.e. from several different follicles) were combined prior to the isolation of granulosa cells on a single Percoll preparation. Having counted the total number of granulosa cells obtained from that patient, 150,000 viable cells were allocated for the triplicate assay of 11β-HSD activity in that multi-follicular pool. Any remaining cells were then combined with cells from the multi-follicular pools of different patients to form a single multi-patient cell pool (where that multi-patient pool contained equal numbers of cells from each patient to a total in excess of 150,000 viable cells at a density of 50,000 viable cells/ml).

In all experiments, cells were cultured for 3 days at 37° C. in an atmosphere of 5% (v/v)

CO₂ in air with a single replacement of medium on the second day of culture.

Radiometric Conversion Assay of 11β-HSD Activity.

Following the 3 day preculture in serum-supplemented medium, 11β-HSD activities were measured by a radiometric conversion assay as previously described.^{12,13,15,37} In brief, cells were incubated on the fourth day of culture in serum-free medium containing 100 nmol/l [1,2,6,7-³H-cortisol (specific activity=10 μCi/nmol) (Amersham International plc, Aylesbury, Bucks., UK) for 4 hours, after which steroids were extracted with chloroform and were resolved by thin layer chromatography (TLC). 11β-HSD activities were calculated as the rate of conversion of [³H]-cortisol to [³H]-cortisone (quantified by liquid scintillation counting), corrected for the specific activity of the substrate, the amount of cellular protein per well and the non-specific rate of generation of [³H]-cortisone. This assay was found to have a finite detection limit of is 10 pmol cortisone formed/mg protein per 4h which equates to the rate of oxidation of cortisol in the presence of 100 μg bovine serum albumin (BSA)(Sigma, UK). For all measures of 11β-HSD activity (i.e. both for individual follicles and for pooled cell measurements), the intra-assay coefficient of variation (CV) for triplicate determinations was 10.87% (mean of 36 independent evaluations) with an inter-assay CV of 12.11% (n=12).

Under these conditions, this radiometric conversion assay provides a measure of the net conversion of (³H-cortisol to (³H]-cortisone by intact granulosa-lutein cells cultured in the presence of a concentration of cortisol (100 nM) known to approximate to that concentration typically measured in follicular fluid (i.e. 200 nM).^{6.7} As such, the assay was not designed to discriminate between the dehydrogenase activities of isoforms of the 11β-HSD enzyme, nor does it measure the gross rate of cortisol oxidation to cortisone since, as with any biochemical reaction, the true rate of the enzyme catalysed reaction will be decreased by the opposing reaction: i.e. the reduction of cortisone to cortisol, catalysed by the 11-ketosteroid reductase (11KSR) activities attributable to one or more 11βHSD isoforms (see conclusions).

Assessment of Oocyte Maturity.

Oocyte maturity was assessed by the observation of oocytes at the time of collection under a dissecting stereo-microscope as described previously.⁸ Oocytes were scored for maturity as follows:

1.0=immature oocytes (i.e. dense/compact cumulus mass with no evidence of germinal vesicle breakdown (GVBD));

2.0 to 2.5=part-mature oocytes (i.e. partially expanded cumulus mass with evidence of GVBD);

3.0=pre-ovulatory oocytes (i.e. expanded cumulus mass with sunburst appearance of the corona and evidence of GVBD); and

3.5 to 4.0=post-mature/luteinized oocytes (i.e. diffuse/dispersed cumulus cells and evidence of oocyte degeneration).

Statistical Analysis of Data.

The 11β-HSD activities of different follicles, each assayed in triplicate, were subjected to one-way analysis of variance (ANOVA) and comparison of these values to the corresponding scores of oocyte maturity was made by Spearman's rank correlation analysis. For three separate patients, the 11β-HSD activities of the multi-follicular pools of cells were compared to the arithmetic means of the activities of the corresponding constituent individual follicles by unpaired t-tests. Likewise, the 11β-HSD activities of the two multi-patient pools of cells were compared to the respective arithmetic means of the activities of the constituent multi-follicular pools for each patient by unpaired t-tests.

While the data for the individual follicles and the multi-patient pools did not conform to normal distributions in every case, the decision to compare the multi-follicular pool and multi-patient pool measurements to the arithmetic means for the appropriate individual follicle/patient values respectively was justified on the basis that equal numbers of cells from each follicle/patient were used to derive the pooled cells in each case. Hence, the expected 11βHSD activity for the multi-follicular/multi-patient pooled cells was calculated to be the simple arithmetic mean of the appropriate individual values, independent of the frequency distribution of the latter data. In all cases, the coefficient of variation (C.V.) for triplicate determinations did not differ significantly between the pooled cell assays and the individual follicle/patient measurements (ANOVA, P>0.05). Moreover, the variation in 11β-HSD activities, both between follicles and between patients, was confirmed to be significantly greater than the variation in triplicate determinations for the corresponding pooled cells (ANOVA, P<0.01). In all cases, P values of less than 5% in a two-tailed test were accepted as statistically significant.

Results

The activity of 11β-HSD in cultured human granulosa-lutein cells recovered from the follicular aspirates of women undergoing oocyte retrieval for the assisted conception protocol of in vitro fertilization and embryo transfer (IVF-ET) has been measured. In such cells 11B-HSD activity modulates sensitivity to the antigonadotrophic actions of cortisol.¹² 11β-HSD activities were measured for each patient in a pool of granulosa cells derived from all of the ovarian follicles aspirated from that patient. In such multi-follicular pools, 11β-HSD activities were found to vary dramatically between different patients and were indeed below the detection limit of the 11β-HSD assay for approximately 40% of patients studied.^{12,13,15} Subsequently it was found that all patients whose granulosa-lutein cells expressed detectable 11β-HSD activity failed to conceive by IVF-ET, whereas the clinical pregnancy rate for those patients with “11β-HSD negative” granulosa-lutein cells was 63%.^{13,15}

11μ-HSD Activities in Individual Follicles.

The 11β-HSD activities of different individual follicles from a given patient (n=3-16 follicles per patient) were found to vary significantly (P<0.05, ANOVA) for each of the 12 patients studied (FIG. 1). However, within each of the 9 patients for which oocyte maturity scores were collated, the 11β-HSD activities of the individual follicles did not correlate to the maturity of the enclosed oocytes, and there was no significant relationship for a total of 34 follicles from 9 patients between the follicular 11βHSD 5 activities and oocyte maturity scores (Spearman's rank correlation: r=−0.178, P>0.05; FIG. 2).

Comparison of 11β-HSD Activities in Individual Follicles with that of the Multi-Follicular Pool of Granulosa-Lutein Cells.

Due to limitations on cell numbers, this second experimental design could only be implemented for cells from three different patients (n=8 to 9 follicles per patient). In each case, the 11β-HSD activity of the multi-follicular pool of cells was found to be significantly lower than the corresponding arithmetic mean of the activities in the appropriate individual follicles FIG. 3). Indeed, in two of these three experiments, the activity of the multi-follicular pool of cells was below the detection limit of the 11β-HSD assay despite contributions of cells from individual follicles that were found to have high 11β-HSD activities when assayed separately (FIG. 3).

Comparison of 11β-HSD Activities in Individual Multi-Follicular Pools of Cells with that of the 11β-HSD Activity of a Multi-Patient Pool of Granulosa-Lutein Cells.

In two independent experiments, the 11β-HSD activity of the multi-patient pool of granulosa-lutein cells was significantly lower than the corresponding arithmetic mean of the activities in the appropriate multi-follicular pool of cells from each patient (Table 2).

Table 2 shows the relationship of the 11β-HSD activity (pmol cortisone/mg protein.4h) of a multi-patient pool of cells to those activities of the constituent multi-follicular pool of cells from each patient in two independent experiments.

(*P<0.05 for the multi-patient pool value versus the corresponding arithmetic mean for the individual patient multi-follicular pools; unpaired t-tests).

TABLE 2

| | 11β-HSD activities | Arithmetic | 11β-HSD activity |
|---|---|---|---|
| Experi- | for individual | mean of | of multi-patient |
| ment No. | patients | individual values | pool |
| 1 | 39.8 | 132.5 | 68.0* |
| | 54.6 | | |
| | 60.8 | | |
| | 65.2 | | |
| | 441.9 | | |
| 2 | <10.0 | 39.3 | 13.6* |
| | 33.3 | | |
| | 35.3 | | |
| | 38.2 | | |
| | 49.1 | | |
| | 69.4 | | |

Conclusions

The main parameter for the selection of oocytes for IVF has been their maturity, as assessed by a scoring system based on the morphological appearance of the cumulus-oocyte complex (COC). It was recently proposed that measurements of ovarian 11β-HSD activities may provide a more objective parameter for assessing the probable outcome of IVF-ET in a given patient.^{13,15} In these Examples the 11β-HSD activity of granulosa-lutein cells varied dramatically (from undetectable to in excess of 500 pmol/mg protein per 4h) in different follicles from a given patient, and 11β-HSD activities did not relate to the maturity of the oocyte contained within each follicle. Previously the ovarian 11β-HSD activity has been measured for each patient in a pool of cells derived from all of the granulosa-lutein cells aspirated from several different follicles. Whereas none of the 101 cycles with detectable ovarian 11β-HSD activity were associated with a clinical pregnancy, the clinical pregnancy rate for the 71 cycles with “11β-HSD negative” cells was 63%.^{13,15} The findings presented here indicate that the ovarian 11β-HSD activities of pooled granulosa-lutein cells are not a simple reflection of the 11β-HSD activities in the individual ovarian follicles; enzyme activities of pooled cells were consistently lower than the mean of the 11β-HSD activities measured in the individual follicles. Indeed, in two of the three patients, the activity of the pooled cells was suppressed to below the detection limit of the radiometric conversion assay despite the inclusion of cells from individual follicles with high enzyme activities. Hence, it is proposed that a pool of human granulosa-lutein cells will only manifest high 11β-HSD activity if all of the constituent follicles are “11β-HSD positive”. Conversely, if one or more follicles contribute granulosa-lutein cells with low 11β-HSD activities, the activity of the multi-follicular pool of cells will be low/undetectable, irrespective of the enzyme activities in the other constituent follicles.

One cannot yet be certain whether the co-culture of cells with low 11β-HSD activities is necessary to suppress the high enzyme activity of cells from different follicles/patients, or whether those cells with low 11β-HSD activity might have or produce a diffusible agent that can inhibit the enzyme activity of neighbouring cells. The findings merely suggest that human granulosa-lutein cells with low 11βHSD activity exert a paracrine action in vitro to suppress glucocorticoid metabolism in cells that would otherwise exhibit moderate to high 11β-HSD activities.

In studies of the regulation of renal 11β-HSD activity, a compound has been identified in urine that can inhibit the activities of both hepatic 11β-HSD and 5β-reductase.²⁰ Since inhibition of these enzymes is a characteristic of glycyrrhetinic acid (the predominant metabolite of glycyrrhizic acid which is itself the active component of liquorice¹⁷), this urinary compound has been said to possess “glycyrrhetinic acid-like activity”. Despite attempts to purify and identify the precise urinary compound(s) responsible for these inhibitory properties, the identity of this agent remains unknown and the compound continues to be referred to as glycyrrhetinic acid-like factor (GALF). While the molecular identity of GALF has proved elusive, it is known that excretion of GALF increases in pregnancy²⁰ which may be a contributing factor in pregnancy-associated hypertension.

In addition to GALF, bile salts,^{4.25} cholesterol,⁴ lanosterol⁴ and a number of steroid hormones^{10,24} have been shown to regulate 11β-HSD activities in both the liver (low affinity, NADP⁺-dependent, type 11β-HSD activity), distal nephron and placenta (high affinity, NAD⁺-dependent, type 2 11β-HSD activity). Hence, it is possible that low levels of ovarian 11β-HSD activity are associated with increased production of sex steroids (e.g. progesterone and oestradiol) by human granulosa-lutein cells in vitro, and that this relationship might form the basis for the paracrine suppression of ovarian 11β-HSD activity indicated by the observations reported here. Indeed, it has been demonstrated that in cultured human granulosa-lutein cells, pregnenolone and progesterone (but not oestrone nor oestradiol) can inhibit 11B-HSD activity³¹.

In view of the indication that ovarian 11β-HSD may be susceptible to paracrine (and possibly autocrine) inhibition, at least in vitro, the findings presented here raise the possibility that an increased probability of conception by IVF-ET is associated not only with low levels of ovarian 11β-HSD activity, but also with increased production of a compound(s) that can inhibit 11β-HSD activity in the multi-follicular pool of human granulosa-lutein cells assayed for each patient. This alternative hypothesis would certainly be consistent with the increased excretion of urinary GALF in pregnancy.²⁰

To date, two isoforms of 11β-HSD have been identified, of which the best characterized is the hepatic isoform, 11βHSD1, which preferentially utilizes NADP(H) as a cofactor, has a supraphysiological Kₘ for its 11β-dehydrogenase activity (Kₘ for cortisol=17 μM; Kₘ for corticosterone≈2 μM) and instead acts predominantly as an 11-ketosteroid reductase (11KSR) converting physiological concentrations of cortisone to cortisol^{9,17,19} (Kₘ for cortisone 140-272 nM). In contrast, in the distal nephron, glucocorticoids are metabolized by a distinct renal isoform of 11βHSD which has a far higher affinity for cortisol and corticosterone (Kₘ=40 nM & 26 nM respectively). This isoform, designated 11β-HSD2, acts predominantly as a high affinity 11β-dehydrogenase and shows an absolute requirement for NAD⁺ as a cofactor.^{24,21,11,32,28} In addition, 11β-HSD2 is capable of metabolizing the synthetic glucocorticoid, dexamethasone^{2,29} and is susceptible to inhibition not only by derivatives of glycyrrhetinic acid, but also by the end-products of 11β-dehydrogenase action^{2,28} (i.e. cortisone and 11-dehydrocorticosterone). 11β-HSD2 has recently been cloned and sequenced^{1,2} and has been shown to be expressed in tissues other than the kidney, including the human ovary.

In initial attempts to characterize those isoforms of 11β-HSD expressed in human granulosa-lutein cells, biochemical evidence has been obtained to suggest the co-expression of at least two distinct isoforms of 11β-HSD, one of which has a high affinity for cortisol similar to that reported for 11β-HSD2.¹⁴ In addition, preliminary Northern blots demonstrate the expression of 11β-HSD1 mRNA in human granulosa cell cultures,¹⁴ supporting the view that there may be more than one isoform of 11β-HSD operating in human granulosa-lutein cells. It is important therefore to acknowledge that the conditions employed for the assay of 11β-HSD activity in these were selected to measure the net rate of inactivation of cortisol to cortisone by intact cells at a concentration of cortisol (100 nM) typical of that measured within follicular fluid.^{6,7} This net rate of cortisol metabolism will obviously depend on which isoform of 11β-HSD predominates in a given culture, and the relative balance between the 11β-dehydrogenase and 11KSR activities of those isoforms of 11β-HSD expressed within a given culture. Indeed, low “11β-HSD activities” in a given follicle or pool of granulosa cells could reflect the presence of cells in which the 11KSR activity of one or more 11βHSD isoforms predominates to such an extent that any cortisone generated by the 11β-dehydrogenase activities is immediately reduced back to cortisol.

In conclusion, these experiments establish that the 11β-HSD activities of individual ovarian follicles can vary dramatically (irrespective of oocyte maturity), and has implicated the paracrine inhibition of ovarian 11β-HSD activity in human granulosa-lutein cells.

### EXAMPLE 3

Follicular fluid, aspirated from the ovarian follicles of women undergoing oocyte retrieval for IVF, has been found to contain at least one compound that, when added to cultured human granulosa-lutein cells in vitro, is capable of inhibiting 11βHSD activities (Table 3). After removal of the sample from the female the follicular fluid was separated from the granulosa-lutein cells, and the latter were cultured separately (for 72 hours) before being brought back into contact with the follicular fluid, in order to test whether there was an 11β-HSD inhibitor present in the fluid. The follicular fluid isolated from patients with low ovarian 11βHSD activities was found to inhibit human granulosa cell oxidation of cortisol to a greater extent than follicular fluid obtained from patients with high ovarian 11βHSD activities (also Table 3). These data are consistent with the hypothesis that human ovarian follicular fluid contains one or more endogenous inhibitors of ovarian 11βHSD activity and that the content of such compounds appears to correlate inversely with the ovarian 11βHSD activities in patients from whom these fluid samples are obtained (i.e. follicular fluids associated with low ovarian 11βHSD activities appear to contain more endogenous 11βHSD inhibitory activity). Since low ovarian 11βHSD activites can be associated with a high probability of conception through IVF-ET, a high concentration of endogenous inhibitor(s) of 11βHSD activity in the ovary may similarly be predictive of conception.

### EXAMPLE 4

It has been confirmed that the compounds mentioned in Example 3, found in ovarian follicular fluid, are also capable of inhibiting renal 11βHSD activities when added to acute (30 minute) incubations of homogenized rat kidneys (Table 4) and that the degree of inhibition is greater in follicular fluids associated with low ovarian 11βHSD activities.

This experiment thus demonstrates the feasibility of using homogenates of rat kidney to assay for endogenous modulators of 11βHSD in biological fluids, such as ovarian follicular fluids.

TABLE 3
Effect of 10% follicular fluid on 11β-HSD in intact granulosa cells.

| | pmoles E formed/4 hours |
|---|---|
| Control | 5.4 ± 0.4 |
| Low Treatment | 2.9 ± 0.2 |
| High Treatment | 3.4 ± 0.3 |

TABLE 4
Effect of 10% follicular fluid on kidney 11β-HSD.

| | pmoles E formed/mg/30 min. |
|---|---|
| Control | 82.8 ± 1.5 |
| Low FF Treatment | 50.7 ± 1.4 |
| High FF Treatment | 70.9 ± 1.0 |

Legend to Tables 3 and 4

11βHSD activities (rates of cortisol oxidation to cortisone [E] over 4 hours and 30 minutes respectively) for (a) cultured human granulosa-lutein cell (Table 3) and (b) rat kidney homogenates (Table 4) treated in vitro with 10% (v/v) follicular fluid samples pooled from patients previously confirmed to have either low or high ovarian 11βHSD activities in the specific IVF cycles in which the follicular fluids had been collected.

### REFERENCES

1. Agarwal A K, Mune T, Monder C & White P C, NAD⁺-dependent isoform of 11β-hydroxysteroid dehydrogenase. Cloning and characterization of cDNA from sheep kidney. Journal of Biological Chemistry 42: 25959-25962 (1994).

2. Albiston A L, Obeyesekere V R, Smith R E & Krozowski Z S, Cloning and tissue distribution of the human 11β-hydroxysteroid dehydrogenase type 2 enzyme. Molecular and Cellular Endocrinology 105: R11-R17 (1994).

3. Benediktsson R, et al, Journal of Endocrinology, 135: 53-58 (1992).

4. Bühler H, Perschel F H, Fitzner R & Hierhoizer K, Endogenous inhibitors of 11β-OHSD: existence and possible significance. Steroids 59: 131-135 (1994).

5. Bush I E, Hunter S A & Meigs R A, Metabolism of 11-oxygenated steroids. Biochemical Journal 107: 239-258 (1968).

6. Dehennin L, Nahoul K & Scholler R, Steroid 21-hydroxylation by human preovulatory follicles from stimulated cycles: a mass spectrometrical study of deoxycorticosterone, 21-hydroxypregnenolone and 11-deoxycortisol in follicular fluid. Journal of Steroid Biochemistry 26: 337-343 (1987).

7. Fateh M, Ben-Rafael Z, Benadiva C A, Mastroianni L Jr & Flickinger G L, Cortisol levels in human follicular fluid. Fertility & Sterility 51: 538-541 (1989).

8. Gregory L, Booth A D, Wells C & Walker S M, A study of the cumulus/corona cell complex in in vitro fertilization and embryo transfer (IVF-ET); a prognostic indicator of the failure of implantation. Human Reproduction 9: 1308-1317 (1994).

9. Lakshmi V & Monder C, Purification and characterization of the corticosteroid 11β-dehydrogenase component of the rat liver 11β-hydroxysteroid dehydrogenase complex. Endocrinology 123: 2390-2398 (1988).

10. Lax E R, Ghraf R & Schreifers H, The hormonal regulation of hepatic microsomal 11β-hydroxysteroid dehydrogenase activity in the rat. Acta Endocrinologica (Copenhagen) 89: 352-358 (1978).

11. Mercer W R & Krozowski Z S, Localization of an 11β-hydroxysteroid dehydrogenase activity to the distal nephron. Evidence for the existence of two species of dehydrogenase in the rat kidney. Endocrinology 130: 540-543 (1992).

12. Michael A E, Pester L A, Curtis P, Shaw R W, Edwards C R W & Cooke B A, Direct inhibition of ovarian steroidogenesis by cortisol and the modulatory role of 11β-hydroxysteroid dehydrogenase. Clinical Endocrinology 38: 641-644 (1993).

13. Michael A E, Gregory L, Walker S M, Antoniw J W, Shaw R W, Edwards C R W & Cooke B A, Ovarian 11β-hydroxysteroid dehydrogenase: potential predictor of conception by in-vitro fertilization and embryo transfer. Lancet 342: 711-712 (18 September 1993).

14. Michael A E, Piercy E C, Stedman B, Antoniw J W, Edwards C R W, Seckl J R & Cooke B A, Evidence for the co-existence of two distinct isoforms of 11β-hydroxysteroid dehydrogenase (11βHSD) in human granulosa-lutein cells. Journal of Endocrinology 140: (Supplement), Abstract OC36 (1994).

15. Michael A E, Gregory L, Piercy E C, Walker S M, Shaw R W & Cooke B A, Ovarian 11β-hydroxysteroid dehydrogenase activity is inversely related to the outcome of in vitro fertilization-embryo transfer treatment cycles. Fertility & Sterility (in press)

16. Monder C, Corticosteroids, receptors and the organ-specific functions of 11β-hydroxysteroid dehydrogenase. FASEB Journal 5, 3047-3054 (1991).

17. Monder C & Lakshmi V, Evidence for kinetically distinct forms of corticosteroid 11β-dehydrogenase in rat liver microsomes. Journal of Steroid Biochemistry 32: 77-83 (1989).

18. Monder C, Stewart P M, Lakshmi V, Valentino R, Burt D & Edwards C R W, Licorice inhibits corticosteroid 11β-dehydrogenase of rat kidney and liver: in vivo and in vitro studies. Endocrinology 125: 1046-1053 (1989).

19. Moore C C D, Mellon S H, Murai J, Siiteri P K & Miller W L, Structure and function of the hepatic form of 11β-hydroxysteroid dehydrogenase in the squirrel monkey, an animal model of glucocorticoid resistance. Endocrinology 133: 368-375 (1993).

20. Morris D J, Semafuko W E B, Latif S A, Vogel B, Grimes C A & Sheff M F, Detection of glycyrrhetinic acid-like factors (GALFs) in human urine. Hypertension 20: 356-360 (1992).

21. Naray-Fejes-Toth A, Watlington CO & Fejes-Toth G, 11β-hydroxysteroid dehydrogenase activity in the renal target cells of aldosterone. Endocrinology 129: 17-21 (1991).

22. Orly and Sato, Cell 17: 295 (1979).

23. Patino and Thomas, J. Exp. 2006, 255: 97 (1990).

24. Pepe G J, Waddel B J, Stahl S J & Albrecht E D, The regulation of transplacental cortisol-cortisone metabolism by estrogen in pregnant baboons. Endocrinology 122: 78-83 (1988).

25. Perschel F H, Buhler H & Hierholzer K, Bile acids and their amidates inhibit 11β-hydroxysteroid dehydrogenase obtained from rat kidney. Pflugers Archives 418: 538-543 (1991).

26. Radwanska E, The role of reproductive hormones in vascular disease and hypertension. Steroids 58: 605-610 (1993).

27. Rundle S E, Funder J W, Lakshmi V and Monder C, The intrarenal localization of mineralocorticoid receptors and 11β-dehydrogenase: immunocytochemical studies. Endocrinology 125: 1700-1704 (1989).

28. Rusvai E & Naray-Fejes-Toth A, A new isoform of 11β-hydroxysteroid dehydrogenase in aldosterone target cells. Journal of Biological Chemistry 268: 10717-10720 (1993).

29. Siebe H, Baude G, Lichtenstein I, Wang D, Buhler H, Hoyer G A & Hierholzer K, Metabolism of dexamethasone: sites and activity in mammalian tissues. Renal Physiology & Biochemistry 16: 79-88 (1993).

30. Tannin et al, Journal of Biological Chemistry, 266: 16653-16658 (1991).

31. Thaventhiran L, Michael A E, Antoniw J W & Cooke B A, Effects of progestins, androgens and oestrogens on 11β-hydroxysteroid dehydrogenase activity in human granulosa-lutein cells. Journal of Endocrinology 144: (Supplement), Abstract P158 (1995).

32. Walker B R, Campbell J C, Williams B C & Edwards C R W, Tissue-specific distribution of the NAD⁺-dependent isoform of 11β-hydroxysteroid dehydrogenase. Endocrinology 131: 970-972 (1992).

33. Webley G E, Luck M R & Hearn J P, Stimulation of progesterone secretion by cultured human granulosa-lutein cells with melatonin and catecholamines. Journal of Reproduction and Fertility 84: 669-677 (1988).

34. Kirk-Othmer Encyclopedia of Chemical Technology, 1982, Vol. 19, pages 631-632).

35. WO-A-89/09088 (Paralog Affinity Chromatography).

36. EP-A-0239400 (Winter).

37. WO-A-94/21815 (Royal Free Hospital School of Medicine, 29 September 1994).

38. Michael A E, Gregory L, Thanenthiran L, Antoniw J W, and Cooke B A, Follicular Variation in Ovarian 11β-hydroxysteroid dehydrogenase (11β-HSD) activities: evidence for the paracrine inhibition of 11β-HSD in human granulosa-lutein cells. Journal of Endocrinology 148:419425 (February 1996).

1. A method of predicting the outcome of, or assessing the chances of success of, in vitro fertilisation (IVF) which method comprises:
(a) determining the activity of a modulator of 11β-hydroxysteroid dehydrogenase (11β-HSD) in one or more biological sample(s) from a female individual; and
(b) predicting, from the activity of 11β-HSD modulator determined, the likelihood or probability of establishing pregnancy in that individual by IVF.

2. A method of predicting the outcome of, or assessing the chances of success of, in vitro fertilisation (IVF) in a female individual, which method comprises:
(a) removing one or more biological sample(s) from a female individual;
(b) determining the activity of a modulator of 11β-hydroxysteroid dehydrogenase (11β-HSD) in the sample: and
(c) predicting, from the level(s) of 11β-HSD modulator determined, the probability or likelihood of establishing pregnancy in that individual by IVF.

3. A method of establishing the likelihood of successful IVF in a female individual which method comprises:
(a1) removing one or more oocyte(s) from a female individual together with a biological sample;
(a2) fertilising the oocyte in vitro:
(b) determining the activity of a modulator of 11β-hydroxysteroid dehydrogenase (11-HSD) in the sample; and
(c) predicting, from the activity of 11β-HSD modulator determined, the likelihood or probability of establishing pregnancy in that individual by IVF.

4. A method of establishing the likelihood of successful IVF treatment in a female individual, which method comprises:
(a) removing one or more oocyte(s) from a female individual together with a biological sample;
(b) determining the activity of a modulator of 11β-hydroxysteroid dehydrogenase (11β-HSD) in the sample;
(c1) predicting, from the activity of 11β-HSD modulator determined, the likelihood or probability of establishing pregnancy in that individual by IVF; and
(c2) fertilising an oocyte from the individual whose 11β-HSD modulator activity is above or below a predetermined threshold level.

5. A method according to any preceding claim wherein the (or each) sample comprises tissue, cells and/or a body fluid from the environment of the oocyte.

6. A method according to claim 5 wherein the sample comprises a follicular aspirate.

7. A method according to any preceding claim wherein the (or each) sample comprises granulosa lutein cells and/or follicular fluid.

8. A method according to any preceding claim wherein the activity of modulator is measured directly by determining the amount or concentration of the modulator.

9. A method according to any preceding claim wherein the modulator is endogenous.

10. A method according to any preceding claim wherein a plurality of biological samples is provided, or removed from the individual, at different times in the cycle, and the activity of modulator determined for each sample.

11. A method according to claim 10 wherein at least 2 samples are removed on successive days.

12. A method according to claim 10 or 11 wherein a decrease (over time) in 11β-HSD activity is indicative of a higher likelihood of pregnancy than a constant, or increase in, level of 11β-HSD activity.

13. A method according to any preceding claim where a plurality of biological samples is provided, or removed from the individual, and the determination of the activity of a modulator comprises:
(i) determining the activity of a modulator of 11β-HSD in at least two of the samples; and
(ii) determining the activity of a modulator of 11β-HSD in a mixture of at least two samples.

14. A method according to claim 13 wherein a comparison of the mean of all the modulator activities determined in (i) with the activity determined in (ii) can indicate:
the presence of a modulator:
whether a modulator, if present, is an inhibitor or agonist of 11β-HSD;
and/or
the amount of modulator present.

15. A method according to any preceding claim wherein the activity of the modulator is to inhibit 11β-HSD.

16. A method according to claim 15 wherein the inhibitory modulator is glycyrrhetinic acid, glycyrrhetinic acid-like factor (GALF), pregnenolone or progesterone.

17. A method according to any preceding claim wherein the female individual is a human female.

18. A method according to any of claims 3 to 17 which further comprises: (d) implanting a fertilized oocyte into the female individual.

19. A method according to any of claims 13 to 18 wherein each sample comprises granulosa lutein cells and/or follicular fluid taken from different parts of an ovary of a female.

20. A method according to any of claims 4 to 19 wherein the predetermined threshold, level is measured as the rate of conversion of ³H-cortisol to ³H-cortisone per mg protein per hour.

21. A method of screening a female population for their suitability for IVF, which method comprises:
(a) screening a population of female individuals seeking treatment for infertility by IVF for activity of modulator(s) of 11β-HSD; and
(b) selecting from the population those individuals who have 11β-HSD modulator activity above or below a predetermined threshold for IVF treatment.

22. A method according to claim 21 which further comprises re-screening individuals with 11β-HSD modulator activity above the predetermined threshold level in subsequent cycles of ovulation.

23. A kit which includes at least one reagent for the detection of an 11β-HSD modulator for use in a method of fertility treatment of a female individual.

24. Use of a reagent for the detection of a 11β-HSD modulator for the prognosis of the likelihood of establishing pregnancy by IVF in a female individual.

25. An identification method, the method comprising:
(a) removing, from a female individual, one or more biological sample(s); and
(b) testing the sample for the presence of an 11β-HSD modulator and, if present determining its identity.

26. A method of increasing the chances of pregnancy, or increasing the likelihood of successful IVF, the method comprising:
(a) optionally, removing from a female individual one or more biological samples;
(b) determining the identity of an inhibitor or agonist present in one of the samples; and
(c) administering, to that female, an amount of that inhibitor or an amount of a compound that interferes with, inhibits or prevents the activity of the agonist (a pregnancy enhancing compound).

27. An inhibitor identified by a method according to claim 26 not previously identified as an 11β-HSD modulator for use in increasing the likelihood of pregnancy.

28. The use of an 11β-HSD inhibitor not previously identified as an 11β-HSD modulator for the manufacture of a pharmaceutical composition for increasing the likelihood of pregnancy in IVF.

29. A method of contraception, the method comprising:
(a) determining the identity of an inhibitor or agonist of 11β-HSD in one or more biological sample(s) from a female individual; and
(b) administering, to that individual, either an amount of the agonist or an amount of a compound that interferes with, inhibits or prevents the activity of the inhibitor (a contraceptive compound).

30. The use of an 11β-HSD agonist or a contraceptive compound (as defined in claim 29) for the manufacture of a contraceptive medicament.

31. A method of identifying a pregnancy enhancing compound or a contraceptive compound not previously identified as an 11β-HSD modulator, the method comprising:
(a) bringing into contact an amount of 11β-HSD and a candidate substance; and
(b) measuring the effect, if any, the substance has on a modulator of 11β-HSD activity;
an inhibition (or decrease) of activity indicating potential pregnancy enhancing properties while an increase in activity indicates potential contraceptive activity.

32. A method of increasing the likelihood or probability of pregnancy, the method comprising:
(a) optionally removing an oocyte from a female individual; and
(b) treating a removed oocyte with an inhibitor of 11β-HSD or a pregnancy enhancing compound identified by a method according to claim 27 or 31.

33. A method according to claim 32 additionally comprising:
(c) fertilising the oocyte before, or after, the treatment in (b); and
(d) implanting the fertilised oocyte into a female individual.
